SEQUENCE LISTING (6 sequences)

SEQ ID NO: 1 - protein, 124 aa, Artificial Sequence
  source: /note=“Description of Artificial Sequence Synthetic polypeptide”
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 2 - protein, 112 aa, Artificial Sequence
  source: /note=“Description of Artificial Sequence Synthetic polypeptide”
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 3 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 107 aa, Homo sapiens [sequence omitted]

SEQ ID NO: 5 - protein, 454 aa, Artificial Sequence
  source: /note=“Description of Artificial Sequence Synthetic polypeptide”
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 219 aa, Artificial Sequence
  source: /note=“Description of Artificial Sequence Synthetic polypeptide”
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0276560 A1
(43) Pub. Date: Sep. 12, 2019

(54) COMPOSITION AND METHOD FOR TREATING ALZHEIMER'S DISEASE

(71) Applicant: Eisai R&D Management Co., Ltd., Tokyo (JP)

(72) Inventors: Andrew SATLIN, New York, NY (US); Tatsuto FUKUSHIMA, Ibaraki (JP)

(73) Assignee: Eisai R&D Management Co., Ltd., Tokyo (JP)

(21) Appl. No.: 16/345,012

(22) PCT Filed: Oct. 26, 2017
(86) PCT No.: PCT/US2017/058587

§ 371 Date: Apr. 25, 2019

Related U.S. Application Data
(60) Provisional application No. 62/413,961, filed on Oct. 27, 2016.
(60) Provisional application No. 62/415,165, filed on Oct. 31, 2016.

(51) Int. Cl. C07K 16/44; A61K 31/542; A61P 25/28

(52) U.S. Cl. CPC C07K 16/44; A61P 25/28; A61K 31/542

(57) ABSTRACT

Methods and combination therapies for treating, preventing, and/or delaying the onset and/or development of Alzheimer's disease using an anti-Aβ protofibril antibody (such as, for example, BAN2401) and N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide and/or a pharmaceutically acceptable salt thereof (Compound X) are provided.

The present application claims the benefit of priority of U.S. Provisional Application No. 62/413,961, filed Oct. 27, 2016, and U.S. Provisional Application No. 62/415,165, filed Oct. 31, 2016, all of which are incorporated herein by reference.

Alzheimer's disease afflicts 1 in 9 elderly individuals, and accounts for dementia in more than 5.2 million Americans and more than 30 million people worldwide. Currently, there is no cure or way to prevent this devastating disease. Histologically, the disease is characterized by neuritic plaques, found primarily in the association cortex, limbic system and basal ganglia. The major constituent of these plaques is amyloid beta peptide (Aβ).

Aβ exists in various conformational states—monomers, oligomers, protofibrils, and insoluble fibrils. Details of the mechanistic relationship between onset of Alzheimer's disease and Aβ production is unknown. However, some anti-Aβ antibodies and beta-secretase (BACE1) inhibitors are undergoing clinical study now as potential therapeutic agents for Alzheimer's disease.

Provided herein are combination therapies for treating, preventing, and/or delaying the onset and/or the development of Alzheimer's disease comprising administering a therapeutically effective amount of anti-Aβ protofibril antibody and a therapeutically effective amount of beta-secretase inhibitor. In some embodiments, the combination therapy inhibits the production of Aβ and/or the toxic oligomeric Aβ. In some embodiments, the combination therapy reduces Aβ and/or the toxic oligomeric Aβ protofibrils in the brain.

Methods, combination therapies, pharmaceutical compositions, and kits for treating, preventing, and/or delaying onset and/or development of Alzheimer's disease using a combination of anti-Aβ protofibril antibody and N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide or a pharmaceutically acceptable salt thereof are described.

As used herein, an anti-Aβ protofibril antibody comprises (a) a heavy chain variable domain comprising the amino acid sequence of SEQ ID NO:1 and (b) a light chain variable domain comprising the amino acid sequence of SEQ ID NO:2. The assignment of amino acids to each domain is, generally, in accordance with the definitions of SEQUENCES OF PROTEINS OF IMMUNOLOGICAL INTEREST (Kabat, et al., 5th ed., U.S. Department of Health and Human Services, NIH Publication No. 91-3242, 1991, hereafter referred to as “Kabat report”).

In some embodiments, the anti-Aβ protofibril antibody comprises a human constant region.

In some embodiments, the human constant region of the anti-Aβ protofibril antibody comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region is chosen from IgG1 and allelic variations thereof. The amino acid sequence of human IgG1 constant region is known in the art and set out in SEQ ID NO:3.

In some embodiments, the human constant region of the anti-Aβ antibody comprises a light chain constant region chosen from K-A-chain constant regions and any allelic variation thereof as discussed in the Kabat report. Any one or more of such sequences may be used in the present disclosure. In some embodiments, the light chain constant region is chosen from K and allelic variations thereof. The amino acid sequence of human K chain constant region is known in the art and set out in SEQ ID NO: 4.

In some embodiments, the anti-Aβ protofibril antibody is BAN2401. mAb158 is a murine monoclonal antibody that was raised to target protofibrils, and BAN2401 is a humanized IgG1 monoclonal version of mAb158. mAb158 has been disclosed in WO2007/108756A1 and Journal of Alzheimer's Disease 43 (2015) 575-588.

BAN2401 is a humanized monoclonal antibody that comprises (a) a heavy chain variable domain comprising the amino acid sequence of SEQ ID NO:1 and (b) a light chain variable domain comprising the amino acid sequence of SEQ ID NO:2. The full length sequence of BAN2401 is set forth in SEQ ID NO:5.

BAN2401 is believed to selectively bind to, neutralize, and eliminate soluble, toxic Aβ aggregates (protofibrils) that are thought to contribute to the neurodegenerative process in Alzheimer's disease. As such, BAN2401 exhibits immunomodulatory effect that may suppress the progression of the Alzheimer's disease. BAN2401 is currently undergoing Phase II clinical trials.

N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide or a pharmaceutically acceptable salt thereof (herein referred to as “Compound X”), represented by the chemical formula (1) shown below, is a Beta-site Amyloid Precursor Protein Cleaving Enzyme 1 (BACE1) inhibitor. See, e.g., U.S. Pat. Nos. 8,158,620 and 8,426,584. Compound X is also known as E2609, or may be also referred to as elenbecestat. By inhibiting BACE, Compound X may decrease Aβ peptides in the brain, potentially improving symptoms and/or slowing the progression of Alzheimer's disease.

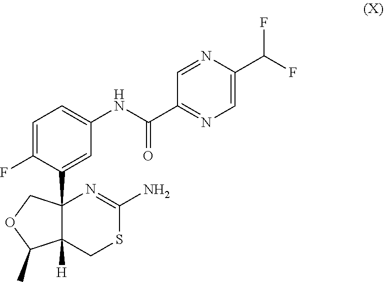 [SMILES: C[C@H]1OC[C@]2(c3cc(NC(=O)c4cnc(C(F)F)cn4)ccc3F)N=C(N)SC[C@H]12]

In some embodiments, Compound X is in the form of a free base.

In some embodiments, methods for preventing, treating, and/or delaying onset and/or development of Alzheimer's disease are provided and comprise administering to a subject in need thereof a therapeutically effective amount of BAN2401 and a therapeutically effective amount of Compound X.

In some embodiments, the subject is an individual who is considered at risk for developing Alzheimer's disease, for example, an individual having at least one family member diagnosed with Alzheimer's disease.

In some embodiments, the subject is an individual who has been diagnosed as having at least one genetic mutation associated with Alzheimer's disease.

In some embodiments, the subject is an individual having at least one mutated or abnormal gene associated with Alzheimer's disease (e.g., an APP mutation, a presenilin mutation, and/or an ApoE4 allele) but who has not been diagnosed with Alzheimer's disease.

In some embodiments, the subject is an individual who is not identified as genetically predisposed to developing Alzheimer's disease.

### BRIEF DESCRIPTION OF DRAWINGS

FIG. 1 shows the amount of Aβ in extracts from the brains of Tg2576 mice.

FIG. 2 shows the effects of the combination treatment in al and β1 frequency bands on EEG recording in Tg2576 hetero mice.

As used herein, the term “preventing” includes, but is not limited to, inhibiting and/or averting one or more biochemical changes, histological changes, and/or behavioral symptoms associated with Alzheimer's disease. Symptoms and pathological changes associated with Alzheimer's disease include, but are not limited to, cognitive decline, increased formation of amyloid plaques, amount of soluble Aβ peptide circulating in biological fluids, accumulation of Aβ peptide in the brain, and abnormalities of memory, problem solving, language, calculation, visuospatial perception, judgment, and behavior.

As used herein, “treatment” or “treating” is an approach for obtaining beneficial and/or desired results, including, but not limited to, clinical results. Non-limiting examples of beneficial and/or desired results include one or more of the following: inhibiting and/or suppressing the formation of amyloid plaques, reducing, removing, and/or clearing amyloid plaques, improving cognition and/or reversing cognitive decline, sequestering soluble Aβ peptide circulating in biological fluids, reducing Aβ peptide (including soluble and deposited) in a tissue (e.g., the brain), inhibiting and/or reducing accumulation of Aβ peptide in the brain, inhibiting and/or reducing toxic effects of Aβ peptide in a tissue (e.g., the brain), decreasing brain atrophy, decreasing one or more symptoms resulting from the disease (e.g., abnormalities of memory, problem solving, language, calculation, visuospatial perception, judgment and/or behavior, inability to care for oneself), increasing the quality of life, decreasing the dose of one or more other medications required to treat the disease, delaying the progression of the disease, altering the underlying disease process and/or course, and/or prolonging survival.

As used herein, the term “treating” is used to describe implementation of the method after the onset of symptoms of Alzheimer's disease, whereas “preventing” is used to describe implementation of the method prior to the onset of symptoms, for example, to patients at risk of Alzheimer's disease.

As used herein, patients at risk of Alzheimer's disease may or may not have detectable disease and may or may not have displayed detectable disease prior to the treatment methods described herein. “At risk” denotes that an individual has one or more measurable parameters (risk factors) that correlate with development of Alzheimer's disease. These risk factors include, but are not limited to, age, sex, race, diet, history of previous disease, presence of precursor disease, genetic (i.e., hereditary) considerations, and environmental exposure. As non-limiting examples, individuals at risk for Alzheimer's disease include those having family history of Alzheimer's disease, those whose risk is determined by analysis of genetic or biochemical markers, those with positive results in a blood test for any signaling proteins present in blood plasma and/or cerebrospinal fluid (“CSF”) known to predict clinical Alzheimer's diagnosis.

As used herein, “delaying” development of Alzheimer's disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of Alzheimer's disease is a method that reduces probability of disease development in a given time frame and/or reduces extent of the disease in a given time frame, when compared to not using the method, including stabilizing one or more symptoms resulting from the disease (e.g., abnormalities of memory, problem solving, language, calculation, visuospatial perception, judgment and/or behavior, inability to care for oneself). Such comparisons may be based on clinical studies, generally using an adequate number of subjects to achieve a statistically significant result. Alzheimer's disease development can be detected using standard clinical techniques, such as standard neurological examination, patient interview, neuroimaging, detecting alterations of levels of specific proteins in the serum or cerebrospinal fluid (e.g., amyloid peptides and/or tau), computerized tomography (CT), magnetic resonance imaging (MRI), and/or positron emission tomography (PET) brain imaging of amyloid or tau. “Development” as used herein may also refer to disease progression that may be initially undetectable and may include occurrence, recurrence, worsening, and/or onset.

As used herein, the terms “effective amount” and “therapeutically effective amount” refer to an amount of a compound or pharmaceutical composition sufficient to product a desired therapeutic effect including, but not limited to, preventing, and/or delaying onset and/or development of at least one disease. The therapeutically effective amount can vary depending upon the intended application, the subject to be treated (including, e.g., weight and age), the disease and its severity, the route and timing of administration, the desired effect (e.g., lower side effect(s)), the dosing regimen to be used, and the formulation and delivery system (if any). In some embodiments, the “therapeutic effective amount” of a drug used in combination with at least one other therapeutic agent may be the same as or different from (either lower or higher) the “therapeutic effective amount” of the drug used individually (i.e., in a monotherapy).

In some embodiments, the combination therapies disclosed herein may comprise lower doses of one or more of the individual therapies than would be necessary if the individual therapies are given alone (i.e., BAN2401 and Compound X monotherapies). This decreased dose may reduce one or more side-effects associated with the therapies. For example, in some embodiments, the same or greater therapeutic benefit is achieved using a smaller amount (e.g., a lower dose or a less frequent dosing schedule) of BAN2401, Compound X, or both, in the combination therapy than the amount(s) generally used for individual therapy. Further as an example, in some embodiments, the use of a small amount of BAN2401, Compound X, or both results in a reduction in the number, severity, frequency and/or duration of one or more side-effects associated with the compounds. As non-limiting examples, the combination therapy may comprise, compared to the doses generally used for individual therapies: (i) lower dose of Compound X and lower dose of BAN2401; (ii) lower dose of BAN2401 and the same dose of Compound X; (iii) lower dose of Compound X and the same dose of BAN2401.

In some embodiments, the combination therapies disclosed herein may comprise higher doses of the individual therapies than would be necessary if the individual therapies were given alone (i.e., BAN2401 and Compound X monotherapies). For example, in some embodiments of the combination therapies, the dose of one of the drugs (BAN2401 and Compound X) is lower than its dose generally used for individual therapy, while the other drug is given at an equal or higher dose than its dose generally used for individual therapy. As non-limiting examples, the combination therapy may comprise (i) higher dose of Compound X and lower dose of BAN2401; or (ii) higher dose of BAN2401 and lower dose of Compound X. In some instances, increasing the dose of one of the drugs while decreasing the dose of the other may have one or both advantages of alleviating the side effects of the drug with lower dose and obtaining the same or greater therapeutic benefit than individual therapies. Further as an example, in some embodiments, the combination therapy may comprise, compared to the dosages generally used for individual therapies, (i) higher dose of Compound X and higher dose of BAN2401; (ii) higher dose of BAN2401 and the same dose of Compound X; or (iii) higher dose of Compound X and the same dose of BAN2401.

In some embodiments, the combination therapy disclosed herein reduces the severity of one or more symptoms associated with Alzheimer's disease by at least 10%, 20%, 30%, 40%, 50%, 60%, 70%, 80%, 90%, or 95% more, as compared to the corresponding symptom in the same subject prior to treatment or as compared to the corresponding symptom in other subjects not receiving the combination therapy. For example, in some embodiments, the administration of the combination of BAN2401 and Compound X results in a reduction of the decline in the measure of cognitive function, such as at least 10%, 20%, 30%, 40%, 50%, 60%, 70%, 80%, 90%, or 95% more, as compared to a control.

In some embodiments, combinations of BAN2401 and Compound X may be administered to a subject in a single dosage form and/or by separate administration of each active agent.

In some embodiments, BAN2401 and Compound X may be formulated into a tablet, pill, capsule, or solution. The formulation of BAN2401 and Compound X may be selected appropriately. In some embodiments, BAN2401, Compound X, or both are formulated into a solution for parenteral administration. In some embodiments, BAN2401 and Compound X may be formulated in segregated regions or distinct caplets of housed within a capsule. In some embodiments, BAN2401 and Compound X may be formulated in isolated layers in a tablet.

In some embodiments, the pharmaceutical composition for treating, preventing, and/or delaying onset and/or development of Alzheimer's disease comprising: a therapeutically effective amount of BAN2401, a therapeutically effective amount of Compound X, and at least one pharmaceutically acceptable carrier.

In some embodiments, BAN2401 and Compound X may be administered as separate compositions and optionally as different forms, e.g., as separate tablets or solutions. For example, in some embodiments, Compound X is administered as once daily oral tablets and BAN2401 is administered as an injection. Further as a non-limiting example, both BAN2401 and Compound X are administered, separately, as oral tablets. Also further as a non-limiting example, both BAN2401 and Compound X are administered, separately, as injections.

In some embodiments, when BAN2401 and Compound X are administered as separate compositions:
the pharmaceutical composition for use in combination with Compound X for treating, preventing, and/or delaying the onset and/or development of Alzheimer's disease comprising a therapeutically effective amount of BAN2401 and at least one pharmaceutically acceptable carrier; and
the pharmaceutical composition for use in combination with BAN2401 for treating, preventing, and/or delaying the onset and/or development of Alzheimer's disease comprising a therapeutically effective amount of Compound X and at least one pharmaceutically acceptable carrier.

In some embodiments, provided herein is a kit comprising a first pharmaceutical composition comprising a therapeutically effective amount of BAN2401, a second pharmaceutical composition comprising a therapeutically effective amount of Compound X, and instructions for use of in treatment, prevention, and/or delaying onset and/or development of Alzheimer's disease.

In some embodiments, BAN2401 and Compound X may be administered simultaneously. In some embodiments, BAN2401 and Compound X may be administered sequentially. In some embodiments, BAN2401 and Compound X may be administered intermittently. The length of time between administrations of BAN2401 and Compound X may be adjusted to achieve the desired therapeutic effect. In some embodiments, BAN2401 and Compound X may be administered only a few minutes apart. In some embodiments, BAN2401 and Compound X may be administered several hours (e.g., about 2, 4, 6, 10, 12, 24, or 36 h) apart. In some embodiments, it may be advantageous to administer more than one dosage of one of BAN2401 and Compound X between administrations of the remaining therapeutic agent. For example, one therapeutic agent may be administered at 1 hour and then again at 11 hours following administration of the other therapeutic agent. In some embodiments, the therapeutic effects of each BAN2401 and Compound X should overlap for at least a portion of the duration, so that the overall therapeutic effect of the combination therapy may be attributable in part to the combined or synergistic effects of the combination therapy.

The dosage of BAN2401 and Compound X may be dependent upon a number of factors including pharmacodynamic characteristics of each agent, mode route of administration, the health of the patient being treated, the extent of treatment desired, the nature and kind of concurrent therapy, if any, the frequency of treatment, and the nature of the effect desired. In some embodiments, BAN2401 may be administered at a dose ranging from about 0.001 mg/kg body weight per day to about 200 mg/kg body weight per day. In some embodiments, BAN2401 may be administered at a dose ranging from 0.001 mg/kg body weight per day to 200 mg/kg body weight per day. In some embodiments, Compound X may be administered at a dose ranging from 5 mg/day to 100 mg/day, 10 mg/day to 75 mg/day, 5 mg/day to 50 mg/day, or 15 mg/day to 50 mg/day. In some embodiments, Compound X may be administered at a dose ranging from about 5 mg/day to about 100 mg/day, about 10 mg/day to about 75 mg/day, about 5 mg/day to about 50 mg/day, or about 15 mg/day to about 50 mg/day. In some embodiments, Compound X may be administered at a dose of 5 mg/day, 10 mg/day, 15 mg/day, 20 mg/day, 25 mg/day, 30 mg/day, or 50 mg/day dosage.

In some embodiments, BAN2401 may be administered at a dose ranging from 2.5 mg/kg to 10 mg/kg, or 5 mg/kg to 10 mg/kg. In some embodiments, BAN2401 is administered at a dose of 10 mg/kg every 2 weeks. In some embodiments, BAN2401 is administered at a dose of 5 mg/kg every 2 weeks. In some embodiments, BAN2401 is administered at a dose of 2.5 mg/kg every 2 weeks. In some embodiments, BAN2401 is administered at a dose of 5 mg/kg every month. In some embodiments, BAN2401 is administered at a dose of 10 mg/kg every month.

In some embodiments, BAN2401 may be administered at a dose ranging from about 2.5 mg/kg to about 10 mg/kg, or about 5 mg/kg to about 10 mg/kg. In some embodiments, BAN2401 is administered at a dose of about 10 mg/kg every 2 weeks. In some embodiments, BAN2401 is administered at a dose of about 5 mg/kg every 2 weeks. In some embodiments, BAN2401 is administered at a dose of about 2.5 mg/kg every 2 weeks. In some embodiments, BAN2401 is administered at a dose of about 5 mg/kg every month. In some embodiments, BAN2401 is administered at a dose of about 10 mg/kg every month.

In some embodiments, each of BAN2401 and Compound X may be administered at a dose regimen as exemplified in Table 1:

Compound X

| BAN2401 | 5 mg/day | 15 mg/day |
|---|---|---|
| 2.5 mg/kg/Biweekly | | |
| 5 mg/kg/Biweekly | | |
| 5 mg/kg/Month | | |
| 10 mg/kg/Biweekly | | |
| 10 mg/kg/Month | | |

In some embodiments, each of BAN2401 and Compound X may be administered at a dose regimen as exemplified in Table 2:

Compound X

| | | |
|---|---|---|
| BAN2401 | 25 mg/day | 50 mg/day |

```
2.5 mg/kg/Biweekly
  5 mg/kg/Biweekly
  5 mg/kg/Month
10 mg/kg/Biweekly
10 mg/kg/Month
```

In some embodiments, a fixed dose of 50 mg of Compound X is administered. In some embodiments, the dosing frequency for BAN2401/placebo infusions is a 2 or a 4-week administration regimen and for Compound X is administered once daily orally in the form of at least one tablet at the highest tolerated dose.

In some embodiments, dose ranges may be varied depending upon the age and weight of the subject being treated and the intended route of administration. In some embodiments, the dose is chosen to improve efficacy and/or maintain efficacy and improve at least one of safety and tolerability. In some embodiments, the dose is chosen to lower at least one side effect and simultaneously improve efficacy and/or maintain efficacy.

In some embodiments, the combinations and methods provided herein may inhibit production of Aβ and/or the toxic oligomeric Aβ. In some embodiments, the combination and methods provided herein may reduce Aβ and/or the toxic oligomeric Aβ protofibrils in the brain.

In some embodiments, the combinations and methods provided herein may result in improved therapeutic efficacy compared to monotherapy with either component alone (i.e., either a BACE1 inhibitor alone or an anti-Aβ protofibril antibody alone). In some embodiments, the combinations and methods provided herein may result in increased safety but equal efficacy (dose sparing, thus reducing adverse events) compared to monotherapy with either component alone.

In some embodiments, the combinations and methods provided herein may follow monotherapy. The combinations and methods provided herein may provide a broader choice to tailor multi-drug regimens to individual patient needs.

In some embodiments, the combination treatments may result in higher reduction of monomeric Aβ, protofibril/oligomer Aβ, or both compared to monotherapy with either component alone.

In some embodiments, the combination treatments may result in greater reduction of number and/or area of Aβ plaque formation in brain compared to monotherapy with either component alone.

In some embodiments, the combination treatments may result in improvement of memory impairment and/or inhibition of hyper-locomotion compared to monotherapy with either component alone.

In some embodiments, the combination treatments may result in improvement of abnormal neuronal viability and/or abnormal synaptic function compared to monotherapy with either component alone.

In some embodiments, the combination treatments may result in improvement of cortical network dysfunction compared to monotherapy with either component alone.

In some embodiments, the combination treatments may result in improvement of upregulated and/or abnormal neuroinflammatory response compared to monotherapy with either component alone.

In some embodiments, the combination treatments may result in inhibition of formation of Aβ and/or tau pathology compared to monotherapy with either component alone.

In some embodiments, the combination treatments may result in improvement of neural and/or glial cell viability compared to monotherapy with either component alone.

In some embodiments, the combination treatments may result in inhibition of altered gene expression by pathologic Aβ compared to monotherapy with either component alone.

### EXAMPLES

### Example 1: Evaluation of Combination Treatment In Vivo in Tg2576 Mice in Biochemical Study

Dosing

To examine the effects of combination treatment of mAb158 (“Compound A”) and Compound X, Tg2576 hetero mice at over 11 month of age were administered Compound A alone (12 mg/kg/week, n=19), Compound X alone (3 mg/kg/day, n=19), or a combination of Compounds A and X (n=19). PBS was used as a vehicle solution for Compound A and a 0.5% methylcellulose solution including 5% 1N HCl (“MC solution”) was used as a vehicle solution for Compound X. Tg2576 mice in a control group (n=20) and wild-type mice (n=15) were administered both vehicle solutions, PBS (6 mL/kg/week) and MC solution (10 mL/kg/day). Dosing of Compound A was done weekly and intraperitoneally and dosing of Compound X was done daily and orally. Tg2576 hetero mice in Compound A group or in Compound X were co-administered MC solution or PBS solution, respectively. The duration of the dosing was 3 months. The mice were sacrificed for the collection of brain, CSF, and plasma samples. The brain was dissected into separate hemispheres after reperfusion. One hemisphere of the brain was frozen in liquid nitrogen and another hemisphere was fixed in 10% phosphate-buffered formalin. The frozen brain samples were utilized for measurement of Aβ species.

Measurement of Aβ in the Brain

The frozen brain hemispheres from Tg2576 mice were homogenized in Tris-Buffered Saline (TBS, Sigma) supplemented with multiple protease inhibitors (cOmplete, Roche) and the homogenized TBS solution was centrifuged at 100,000 g for 1 hr at 4° C. The supernatant (soluble extract) and precipitate were separated from the TBS solution after centrifugation and the precipitates were sequentially homogenized with 70% formic acid (as insoluble extract). For each brain, the concentrations of Aβ in both soluble and insoluble extracts were measured by Aβ ELISA (human/rat Aβ(40)/(42) ELISA kit, Wako). In the levels of Aβ40/42 in each extract, the statistical difference between the vehicle control group and the combination group was analyzed primarily by Student t-test using the GraphPad Prism (GraphPad Software, Inc.). Based on the significant difference between the vehicle control group and the combination group, the comparisons between the combination group and the Compound A (alone) group or the Compound X (alone) group were performed by multiple Dunnett's test.

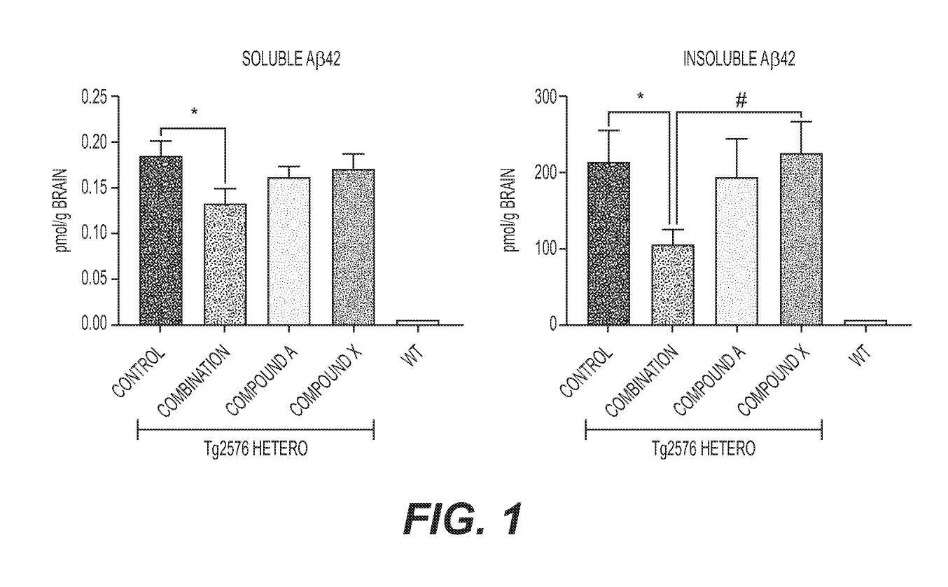

FIG. 1 shows the results of measuring Aβ42 in the soluble and insoluble brain extracts from Tg2576 mice.

Combination treatment with Compound A and Compound X resulted in significant reduction of the level of Aβ42 compared with vehicle treatment (p=0.042). In the insoluble extract, the combination treatment resulted in significant reduction of the level of Aβ42 compared with vehicle treatment (p=0.035). Statistical differences between control group and the combination group and between the combination group and Compound X alone group are indicated in the figure, where * indicates p<0.05 and ^{#} indicates p<0.1.

### Example 2: Evaluation of Combination Treatment In Vivo in Tg2576 Mice in Biochemical, Pathological, and EEG Measurement Studies

Dosing

To examine the effects of combination treatment of Compound A and Compound X, Tg2576 mice at over 11 months of age were administered with one of Compound A alone (12 mg/kg/week, n=7), Compound X alone (3 mg/kg/day, n=8), or a combination of Compounds A and X (n=9). Dosing of Compound A was done weekly and intraperitoneally and dosing of Compound X was done daily and orally. A PBS solution was used for administration of Compound A and a 0.5% methylcellulose solution, including 5% 1N HCl (MC solution), was used for administration of Compound X. Tg2576 hetero mice in control group (n=8) were administered with both vehicle solutions, PBS (6 mL/kg/week) and MC solution (10 mL/kg/day). The mice in Compound A group or in Compound X were co-administered MC solution or PBS solution, respectively. The duration of dosing was 3 months. During the last week of the dosing, the mice were functionally evaluated by analysis of the electroencephalogram (EEG) recordings.

EEG Measurement

Under inhalant anesthesia using isoflurane, Tg2576 mice were held in a stereotaxic apparatus, and their skulls were exposed for implantation of the recording electrodes of EEG and electromyogram (EMG). Four EEG electrodes were inserted into the skull. Two of the electrodes were placed at the positions on the right side [Anterior-Posterior (AP)=1.1 mm/Lateral (L)=1.3 mm and AP=−4.0 mm/L=1.3 mm], and the remaining two were on the left side [AP=1.1 mm/L=−1.3 mm and AP=−4.0 mm/L=−1.3 mm]. The EMG electrodes for myoelectric potential were subsequently implanted in the right and left cervical skeletal muscles.

The recording of EEG and EMG was performed on the mice placed in cages for measuring EEG and EMG. The EEG and EMG signals were led out on line from the electrodes implanted in Tg2576 mice and amplified through three-channel biopotential recording system (Pinnacle Technology, Inc.), and recorded in a hard disc using a data acquisition software (Sirenia Acquisition, Pinnacle Technology, Inc.).

Analysis of EEG power spectra was performed using the SleepSign software (KISSEI COMTEC CO., LTD.). Recorded EEG data from each mouse was analyzed by fast Fourier transformation (FFT) to obtain EEG power value (raw EEG power) in each frequency.

The raw EEG powers were divided into 8 frequency bands in the range between 1 and 100 Hz. The 8 frequency bands were constructed from δ (1-4 Hz), θ (4-8 Hz), α1 (8-11 Hz), α2 (11-13 Hz), β1 (13-22 Hz), β2 (22-30 Hz), γ1 (30-48 Hz), and γ2 (52-100 Hz). In every dosing group, the EEG power was summarized in each frequency band, and compared among the groups.

Statistical analysis was performed using the GraphPad Prism (GraphPad Software, Inc.). Firstly, statistical analysis by Student's t test was performed for the EEG powers between vehicle control group and the combination group in each δ, θ, α1, α2, β1, β2, γ1, and γ2 frequency band. In the case that the EEG power in a frequency band showed significant difference by the first statistical analysis, the differences of EEG powers between the combination group and the Compound A alone or Compound X alone group were analyzed by one-way analysis of variance (ANOVA) followed by Fisher's Least Significant Difference test. The combination group had statistically significant increasing EEG powers compared to vehicle control group in al and β1. Further statistical analysis showed that EEG powers in combination group were significantly increased compared to Compound A group (p=0.0009 in α1 and 0.0106 in β1, respectively) and had a trend to increase compared to Compound X group (p=0.0512 in α1).

It was reported that resting stage EEG features in AD patients are characterized by an increase of widespread delta and theta activity as well as a reduction in posterior alpha and beta activity and increase of slow EEG power coupled with a decrease in alpha activity is linked to cognitive performance decline in MCI compared to healthy subjects (Electroencephalogram and Alzheimer's Disease: Clinical and Research Approaches (A. Tsolaki, et al., International Journal of Alzheimer's Disease, 2014); Electroencephalographic Rhythms in Alzheimer's Disease (R. Lizio et al., International Journal of Alzheimer's Disease, 2011). The effects of the combination treatment in AD mouse model presented here indicate that the combination treatment may improve the EEG abnormalities in AD patients.

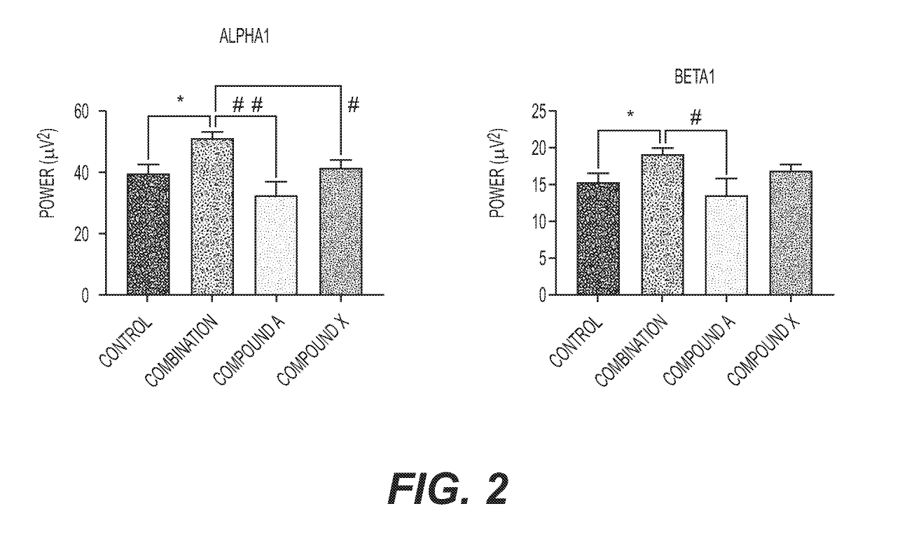

FIG. 2 shows the effects of the combination treatment in α1 and β1 frequency bands on EEG recording in Tg2576 hetero mice. Statistical differences between vehicle control group and combination group and between combination group and Compound A alone group are indicated in the figure, where * indicates p<0.05, and ^{#} and ^{##} indicate p<0.1 and p<0.05, respectively.

### Example 3: Formulations

In some embodiments, Compound X can be formulated into a solid dosage form according to Example 1 of WO2016/056638. Specifically, 2500 mg of Pharmacological Compound 1, 2285 mg of lactose (DFE Pharma Corp.), 330 mg of low-substituted hydroxypropyl cellulose (Type LH21, Shinetsu Chemical Co., Ltd.) and 165 mg of hydroxypropyl cellulose (Type SL, Nippon Soda Co., Ltd.) are mixed in a mortar. A suitable amount of aqueous ethanol (30% w/w) is added to the resulting mixture followed by wet-granulating in the mortar. After drying the resulting granules using a constant temperature bath, the granules are sized using a sieve having 1 mm openings. 33 mg of low-substituted hydroxypropyl cellulose (Type LH21, Shinetsu Chemical Co., Ltd.) and 11 mg of sodium stearyl fumarate (JRS Pharma Corp.) are added per 1056 mg of the sized granules and mixed in a vial. The resulting mixture is compressed at 9 kN using a single-punch tableting machine to obtain tablets having a diameter of 6.5 mm and weight of 110 mg.

BAN2401, on the other hand, can be formulated by conventional method into a liquid dosage form comprising, for instance, sodium citrate, sodium chloride, and polysorbate 80 with a pH of about 5. In some embodiments, the formulation can comprise 10 mg/mL BAN2401, 25 mM sodium citrate, 125 mM sodium chloride, and 0.2% (w/v) polysorbate 80, and have a pH 5.7.

### Example 4: A Placebo-Controlled, Double-Blind, Double-Dummy, Factorial Design, 24 Month Study to Evaluate Safety and Efficacy of Compound X, BAN2401 and the Combination of Compound X and BAN2401 in Subjects with Early Alzheimer's Disease

Study Design

This study is a multicenter, double-blind, factorial design, double-dummy, placebo controlled, study in subjects with Early Alzheimer's disease. The study incorporates a double dummy design using placebos matched to intravenous infusions of BAN2401 and to oral tablets of Compound X, to enable complete blinding across both monotherapy and co-administration arms of the study. All subjects receive both intravenous infusions and orally administered tablets.

The dose and dosing frequency for BAN2401/placebo infusions are the highest of well-tolerated regimens selected for Phase 3 development based on the results of the ongoing Study BAN2401-G000-201, which is currently exploring both 2 and 4-week administration regimens. Compound X is administered as once daily oral tablets at the highest tolerated dose from ongoing study. A total of 3064 subjects is randomized across 4 treatment groups, all of which are proposed to administered with/without currently approved and stable treatments for Alzheimer disease:
Arm A (Placebo): Placebo administered daily orally and intravenously (IV) as infusions every 2 (or 4) weeks,
Arm B (Compound X Monotherapy): Compound X administered daily orally and placebo administered as IV infusion every 2 (or 4) weeks,
Arm C (BAN2401 Monotherapy) BAN2401 administered as IV infusion every 2 (or 4) weeks and placebo administered daily orally
Arm D (Co-Administration): BAN2401 administered as IV infusion for 2 doses that are 2 (or 4) weeks apart, with placebo administered daily orally until the planned 3rd dose of BAN2401. From the 3rd dose of BAN2401, BAN2401 is administered as IV infusion every 2 (or 4) weeks with Compound X administered daily orally.

Subjects are to be randomized to a fixed 1:1:1:1 schedule across the 4 treatment arms of the study. Subject randomization is stratified according to ApoE genotype, concurrent Alzheimer's disease medication use, and severity of Alzheimer's disease at the time of randomization (i.e., mild cognitive impairment due to Alzheimer's disease/prodromal vs. mild dementia).

Inclusion Criteria

Diagnosis

Mild Cognitive Impairment due to Alzheimer's disease—intermediate likelihood/Prodromal Alzheimer's disease:
1. Meet the National Institute of Aging—Alzheimer's Association (NIA-AA) core clinical criteria for mild cognitive impairment due to Alzheimer's disease—intermediate likelihood;
2. Have a Clinical Dementia Rating (CDR) score of 0.5 and CDR Memory Box score of 0.5 or greater at Screening and Baseline; and
3. Report a history of subjective memory decline with gradual onset and slow progression over the last 1 year before Screening.

Mild Alzheimer's Disease Dementia:
1. Meet the NIA-AA core clinical criteria for probable Alzheimer's disease dementia; and
2. Have a CDR score of 1.0 and a Memory Box score of 0.5 or greater at Screening and Baseline.

Key Inclusion Criteria that must be met by ALL Subjects:
1. Positive biomarker for brain amyloid pathology as indicated by at least one of the following:
   a) PET assessment of imaging agent uptake into brain; a historical amyloid positive PET scan may be used if conducted within 12 months of Screening and provided that the scan and result are considered acceptable by the central PET reading group
   b) CSF assessment of Aβ(1-42)
2. Mini Mental State Examination score equal to or greater than 22 at Screening and Baseline.

Study Treatment(s)

The study incorporates a double dummy design, using placebos matched to intravenous infusions of BAN2401 and to oral tablets of Compound X, to enable complete blinding across both monotherapy and co-administration arms of the study. All subjects receive both intravenous infusions and orally administered tablets.

The dose and dosing frequency for BAN2401/placebo infusions are selected from the most effective and well-tolerated regimen based on the results from the ongoing study BAN2401-G000-201, which is currently exploring both 2- and 4-week administration regimens.

Compound X is administered as once daily oral tablets at the highest dose hypothesized to be optimally safe and effective based on data from ongoing study.

Compound X is supplied as tablets of 50 mg dose strength. Placebo tablets to match Compound X are of identical appearance. Each subject receives one tablet of Compound X or placebo, to be administered orally quaque die (“QD”) in the morning with food.

BAN2401 drug product is formulated as a sterile, non-pyrogenic liquid for intravenous administration. Each vial contains 5 mL of a 100 mg/mL solution of BAN2401 in isotonic buffer. BAN2401 is administered in normal saline as IV infusion. BAN2401 must be administered with an infusion system containing a terminal 0.22 μM in-line filter. BAN2401 is administered on a mg/kg basis or placebo. All subjects receive either biweekly or monthly infusions.

For example, BAN2401 is administered at a dose of 2.5 mg/kg/biweekly, 5 mg/kg/biweekly, 10 mg/kg/biweekly 5 mg/kg/month, or 10 mg/kg/month.

In previous studies with BAN2401/placebo, infusion reactions were common AEs which typically occurred on the first infusion, and can be avoided or minimized using prophylactic medication administered prior to subsequent infusions. Therefore, initiation of Compound X treatment is delayed in the co-administration arm to start on the same day as the 3rd intravenous infusion of BAN2401 to avoid possible confounding between determination of adverse events from infusion reactions related to BAN2401 and adverse events associated with co-administration.

To ensure full blinding, for subjects assigned to Arm D (Co-Administration), the initial two infusions of BAN2401 are administered intravenously while subjects take placebo tablets orally QD. At the time of the third infusion, subjects start Compound X to be taken orally QD and continue for the duration of the study.

Efficacy Assessments

The CDR/Clinical Dementia Rating Sum of Boxes, Mini Mental State Examination, Functional Assessment Questionnaire (FAQ) and Modified the Alzheimer's Disease Assessment Scale-Cognitive Subscale (ADAS-Cog14) are well-established clinical tools for use in the assessment of Alzheimer's Disease.

Disease progression is defined as an increase from baseline by at least 0.5 points on the CDR scale on 2 consecutive scheduled visits at which CDR is undertaken. For subjects with CDR of 0.5 at Baseline, disease progression is indicated by CDR of 1 and greater. For subjects with CDR of 1.0 at Baseline, disease progression is indicated by CDR of 2 and greater.

What is claimed is:

1. A method of treating Alzheimer's disease comprising administering to a subject in need thereof a therapeutically effective amount of an anti-Aβ protofibril antibody and a therapeutically effective amount of N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide and/or a pharmaceutically acceptable salt thereof,
wherein the antibody comprises (a) a heavy chain variable domain comprising an amino acid sequence of SEQ ID NO:1 and (b) a light chain variable domain comprising an amino acid sequence of SEQ ID NO:2,
wherein the antibody is administered at a dose ranging from 2.5 mg/kg to 10 mg/kg,
wherein N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide and/or a pharmaceutically acceptable salt thereof is administered at a dose ranging from 5 mg/day to 50 mg/day.

2. The method of claim 1, wherein N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide and/or a pharmaceutically acceptable salt thereof is administered at a dose ranging from 15 mg/day to 50 mg/day.

3. The method of claim 1, wherein N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide and/or a pharmaceutically acceptable salt thereof is administered at a dose of 50 mg/day.

4. The method of claim 1, wherein the antibody is administered at a dose ranging from 5 mg/kg to 10 mg/kg.

5. The method of claim 1, wherein the antibody is administered at a dose of 10 mg/kg.

6. The method of claim 1, wherein the antibody is administered every 2 weeks.

7. The method of claim 1, wherein the antibody is administered every month.

8. The method of claim 1, wherein (a) the heavy chain constant region further comprises an amino acid sequence of SEQ ID NO:3 and (b) the light chain constant region further comprises an amino acid sequence of SEQ ID NO:4.

9. The method of claim 1, wherein N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide is in the form of a free base.

10. A pharmaceutical composition for use in treating of Alzheimer's disease comprising an anti-Aβ protofibril antibody and N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide or a pharmaceutically acceptable salt thereof,
wherein the antibody comprises (a) a heavy chain variable domain comprising an amino acid sequence of SEQ ID NO:1 and (b) a light chain variable domain comprising an amino acid sequence of SEQ ID NO:2.

11. The pharmaceutical composition of claim 10, wherein (a) the heavy chain constant region further comprises an amino acid sequence of SEQ ID NO:3 and (b) the light chain constant region further comprises an amino acid sequence of SEQ ID NO:4.

12. The pharmaceutical composition of claim 10, wherein N-[3-((4aS,5R,7aS)-2-amino-5-methyl-4a,5,7,7a-tetrahydro-4H-furo[3,4-d][1,3]thiazin-7a-yl)-4-fluorophenyl]-5-difluoromethylpyrazine-2-carboxamide is in the form of a free base.
